(19) United States
(12) Patent Application Publication
(10) Pub. No.: US 2006/0276800 A1
(43) Pub. Date: Dec. 7, 2006

(54) Intervertebral disc replacement and surgical instruments therefor

(75) Inventors: Casey K. Lee, Florham Park, NJ (US); George Makris, West Orange, NJ (US)

Correspondence Address: MILES & STOCKBRIDGE PC, 1751 PINNACLE DRIVE, SUITE 500, MCLEAN, VA, 22102-3833, US

(73) Assignee: Nexgen Spine, Inc.

(21) Appl. No.: 11/340,505

(22) Filed: Jan. 27, 2006

Related U.S. Application Data
(60) Provisional application No. 60/647,002, filed on Jan. 27, 2005.

(51) Int. Cl. A61B 17/58

(52) U.S. Cl. 606/90; 606/96; 606/102

(57) ABSTRACT

A suite of surgical tools for use in implantation of an intervertebral disc prosthesis is provided. The surgical tools include an external alignment frame (EAF), an annulus incision guide (AIG), a depth gauge, a positioning/alignment block, a disc space distractor, an endplate drill guide (EDG), and a vertebral endplate cutter (VEC), any or all of which can be used by a surgeon in a procedure for preparing an implantation site by excising all or part of a degenerated intervertebral disc and implanting an intervertebral disc prosthesis in the site so prepared.

### CROSS REFERENCE TO RELATED APPLICATIONS

This application claims the benefit of U.S. Provisional Application No. 60/647,002, filed Jan. 27, 2005, the entire disclosure of which is incorporated herein by reference.

### BACKGROUND OF THE INVENTION

1. Field of the Invention

This invention relates to surgical instruments used in implantation of intervertebral disc prostheses and to methods of implanting intervertebral disc prostheses.

2. Background Art

The most common pathologic condition causing chronic low back pain and neck pain is degeneration of an intervertebral spinal disc. Such degenerative disc disease (DDD) has been treated by both surgical and non-surgical procedures. When non-operative treatment fails to relieve chronic disabling pain, disc excision, decompression, and/or spinal fusion have been surgical procedures commonly performed on patients with degenerative disc disease. More recently, replacement of a degenerated disc with an artificial disc prosthesis has become an available choice that may reduce or eliminate pain and may restore at least some of the normal disc function.

Disc replacement surgery involves complex and delicate surgical technique. Successful disc replacement surgery depends on many factors, including, e.g., proper positioning and alignment of the prosthesis, congruent contact surface area between the prosthesis and the adjacent vertebrae, and immediate post-operative prosthetic stability within the disc space. It has been recognized that the clinical results are closely correlated with proper positioning of the disc prosthesis in the disc space. Furthermore, a disc prosthesis that is not securely stabilized within the intervertebral disc space can produce post-operative accelerated disc degeneration and formation of osteophytes. Consequently, for a successful outcome, the surgical procedure and surgical instruments are important factors, as well as the design of the disc prosthesis. In particular, the surgical procedure should be simple and the surgical instruments should be designed and fabricated for reliability and convenience in use.

### SUMMARY OF THE INVENTION

According to the invention, a suite of surgical tools is provided to achieve accurate positioning and sizing of the surgical removal of appropriate amounts of the annulus and nucleus of the intervertebral disk, accurate measurement of the intervertebral cavity so prepared, accurate and convenient preparation of seats for the prosthesis in the endplates of the adjacent vertebrae, and convenient insertion of a prosthesis into the prepared location.

To this end the invention provides an external alignment frame (EAF), an annulus incision guide (AIG), a depth gauge, a positioning/alignment block, a disc space distractor, an endplate drill guide (EDG), and a vertebral endplate cutter (VEC), any or all of which can be used by a surgeon in a procedure for preparing an implantation site by excising all or part of a degenerated intervertebral disc and implanting an intervertebral disc prosthesis in the site so prepared.

### BRIEF DESCRIPTION OF THE DRAWINGSS

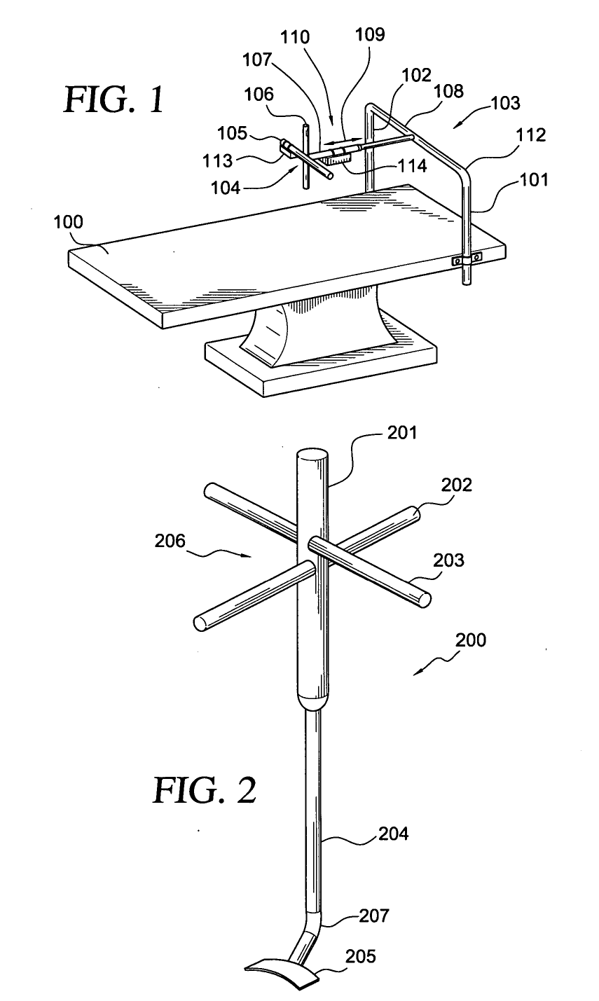

FIG. 1 illustrates an external alignment frame according to the invention.

FIG. 2 illustrates an annulus incision guide (AIG).

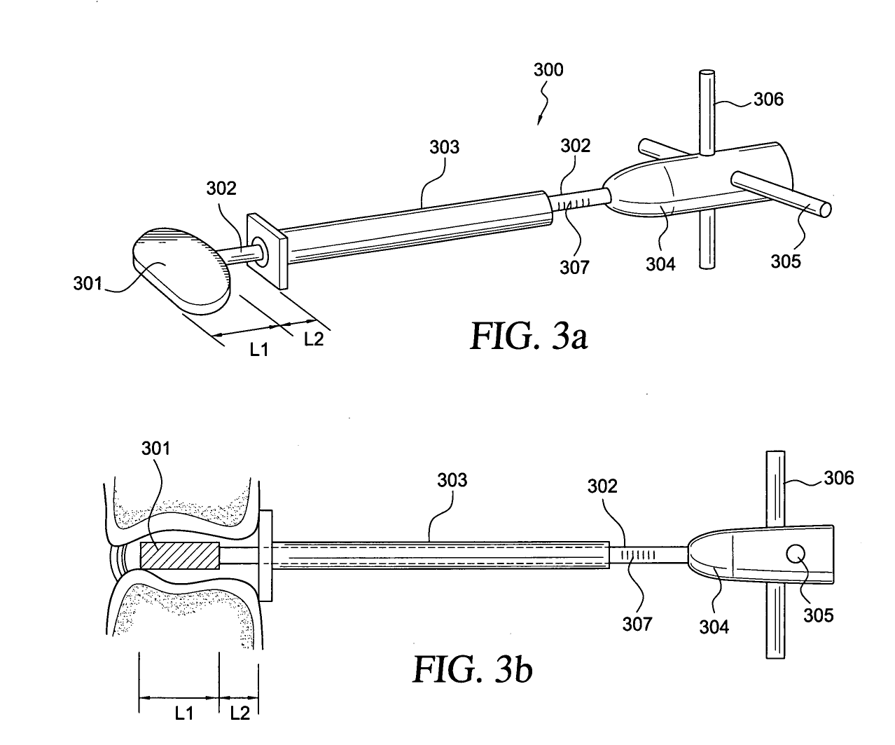

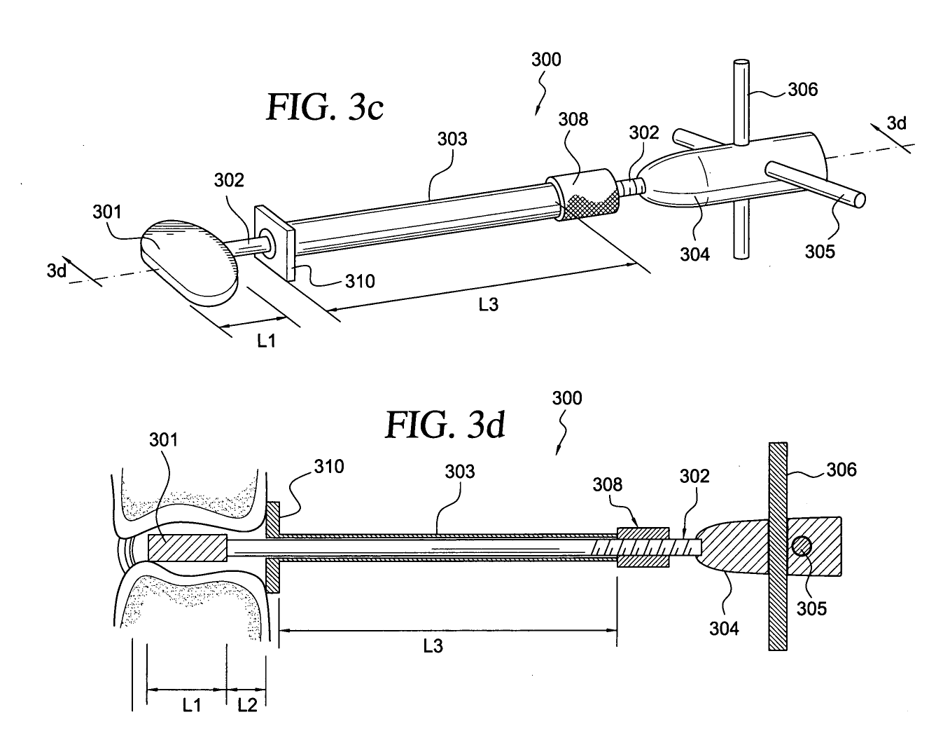

FIG. 3a is a perspective view of a depth gauge according to the invention.

FIG. 3b is a side elevational partial cross-sectional view showing the use of the depth gauge of FIG. 3a.

FIG. 3c is a generally perspective view of an alternate embodiment of the depth gauge of FIG. 3a.

FIG. 3b is a side elevational cross-sectional view showing the use of the depth gauge of FIG. 3c.

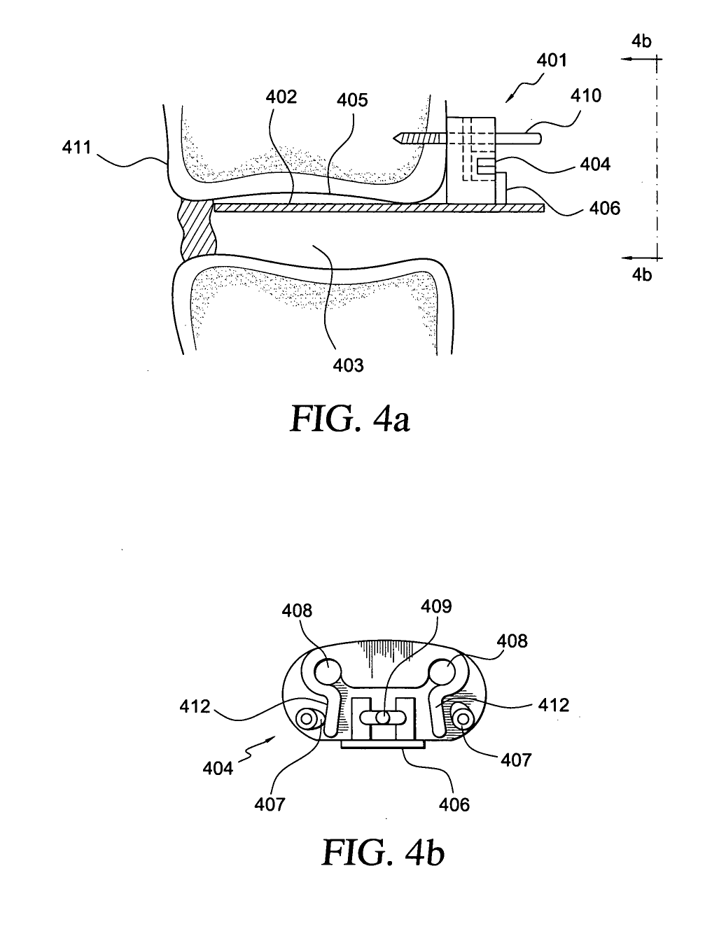

FIG. 4a is a side elevational view of a position/alignment block used with the vertebral endplate cutter (VEC) of the invention.

FIG. 4b is an end elevational view of the position/alignment block taken in the direction indicated by the line 4b-4b in FIG. 4a.

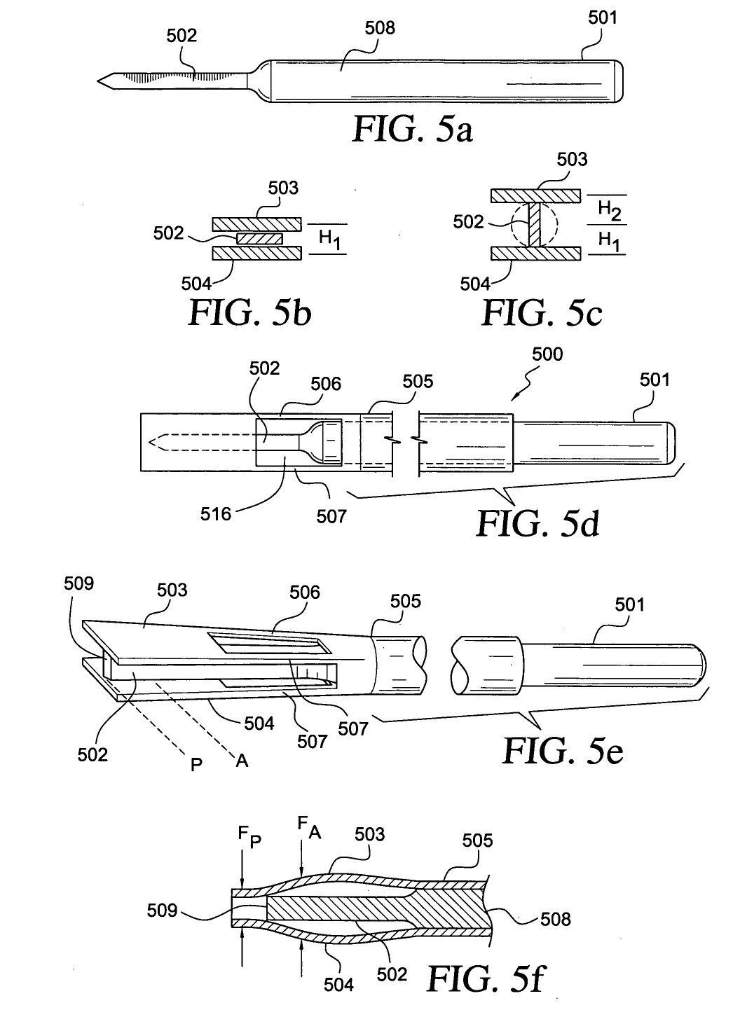

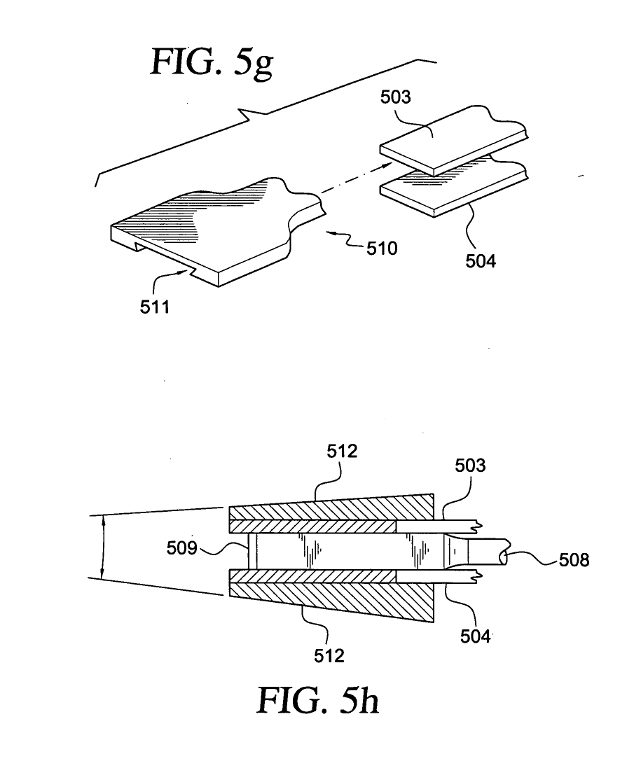

FIG. 5a is a side elevational view of the rotating distractor element of the disc space distractor of the invention.

FIG. 5b is an end view of the disc space distractor of the invention in its collapsed position as inserted into the intervertebral disc space after removal of all or a portion of a degenerated intervertebral disc.

FIG. 5c is an end view of the disc space distractor of the invention in its expanded position as operated to distract adjacent vertebrae.

FIG. 5d is a plan view of the disc space distractor of the invention in its expanded position as operated to distract adjacent vertebrae.

FIG. 5e is a perspective view of the disc space distractor of the invention in its expanded position as operated to distract adjacent vertebrae.

FIG. 5f is a detail cross-sectional view of the distal end of the disc space distractor of the invention in its collapsed state.

FIG. 5g is a detail perspective view of the distal end of the disc space distractor of the invention showing the use of a parallel spacer block.

FIG. 5h is a detail cross-sectional view of the distal end of the disc space distractor of the invention showing the use of angled spacer blocks.

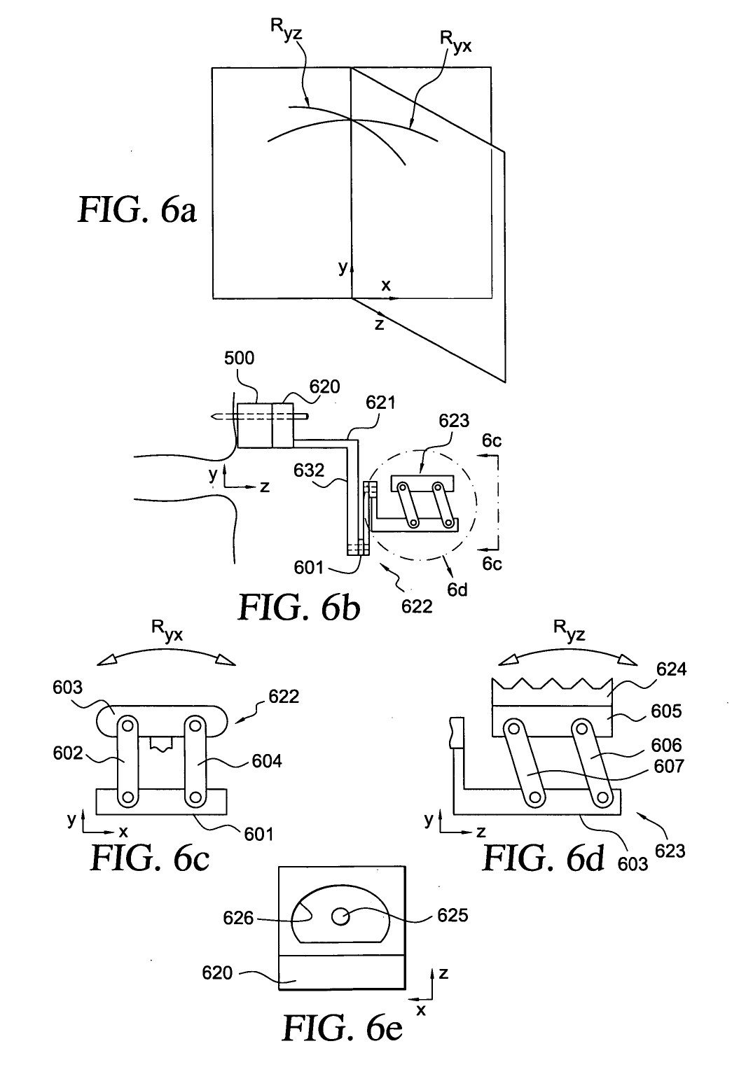

FIG. 6a is a schematic illustration showing the planes and axes used in describing the action of the endplate drill guide (EDG) and vertebral endplate cutter (VEC) of the invention.

FIG. 6b is a side elevational view of the endplate drill guide (EDG) of the invention.

FIG. 6c is a detail end view of the endplate drill guide (EDG) of FIG. 6b, taken in the direction indicated by line 6c-6c in FIG. 6b, showing the operation of the linkage in producing motion of the cutter along R_{yx}.

FIG. 6d is a detail side elevational view of the endplate drill guide (EDG) of FIG. 6b, showing the operation of the linkage in producing motion of the cutter along R_{yz}.

FIG. 6e is a detail plan view of the endplate drill guide (EDG) of FIG. 6b, showing the profile limiting template and pin of the endplate drill guide.

FIG. 6f is a perspective view of the endplate drill guide (EDG) of the invention.

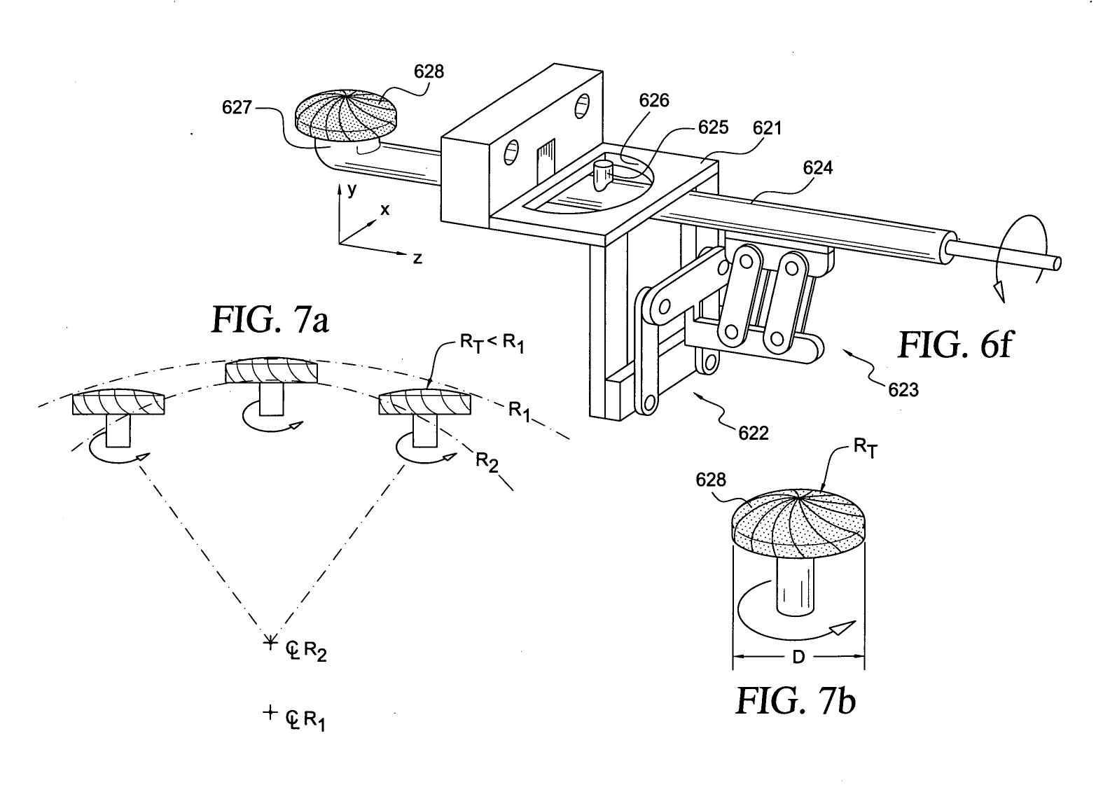

FIG. 7a is a lateral elevational view of the milling cutter of the invention showing its motion along a radius determined by the endplate drill guide and illustrating the radius of the milled seat in the vertebral endplate produced by the combined cross sectional profile of the cutter and its motion.

FIG. 7e is a perspective view of the milling cutter used with the vertebral endplate cutter (VEC) of the invention.

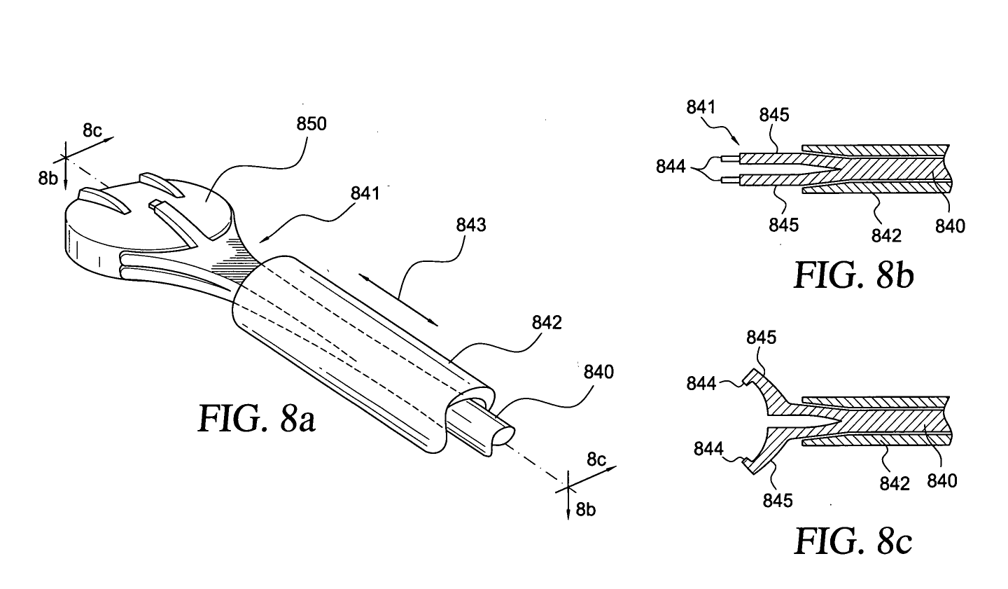

FIG. 8a is a perspective view of the disc prosthesis holder of the invention holding a disc prosthesis prior to implantation.

FIG. 8b is a detail cross-sectional elevational view of the distal end of the disc prosthesis holder of FIG. 8a taken in the direction indicated by the arrow 8b in FIG. 8a.

FIG. 8c is a detail cross-sectional plan view of the distal end of the disc prosthesis holder of FIG. 8a taken in the direction indicated by the arrow 8c in FIG. 8a.

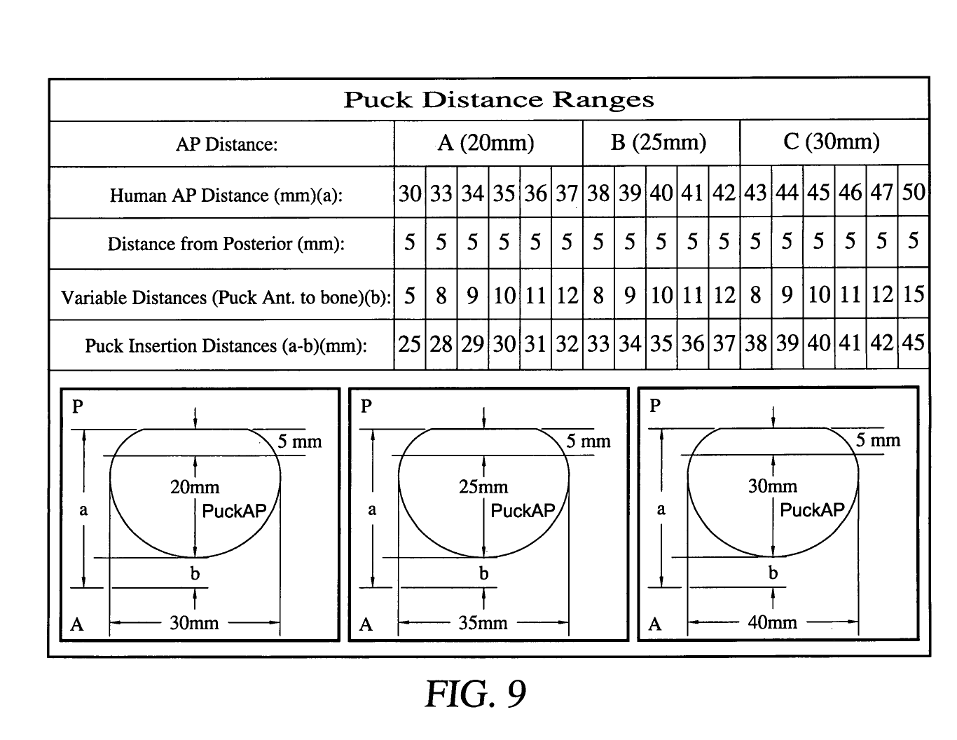

FIG. 9 is a chart indicating possible sizes of the trial prosthesis size-determining element for use with the depth gauge of the invention.

### DETAILED DESCRIPTION OF THE INVENTION AND PREFERRED EMBODIMENTS

Replacement of a degenerated intervertebral disc using the instruments of the invention may be conducted according to the surgical procedure described below.

The spinal motion segment containing the degenerated disc to be replaced with a prosthesis is preoperatively evaluated by noninvasive visualization, e.g., preferably by a computerized tomography (CT) scan, to measure the maximum diameter of the anterior-posterior (AP) axis and the right-left axis of the relevant intervertebral region. Based on this information, an initial estimate of the most likely size of the disc prosthesis to be implanted is made.

The surgical procedure using the instruments of the invention will be described with reference to the instruments, which are illustrated in the accompanying drawings and described in more detail below.

The patient is positioned in the supine posture on the operating table. An external alignment frame (EAF) (FIG. 1) is attached to the operating table and a roentgenogram for the anterior-posterior view of the spine is obtained to confirm the midline position and alignment of the external alignment reference bar (FIG. 1) over the spine.

The spinal column is exposed through the anterior approach. Once the disc and adjoining vertebral bodies are adequately exposed, an annulus incision guide (AIG) (FIG. 2) is placed on the anterior aspect of the disc and the handle of the annulus incision guide (AIG) is aligned to the external alignment frame (EAF), thereby to determine the midline positioning, proper alignment, and size of the annulotomy. The degenerated disc is then progressively removed by a conventional surgical procedure. As the disc is removed, a depth gauge (DG) (FIG. 3) is used periodically to determine the depth of the disc excision. The depth gauge (DG) may be initially set to a predetermined depth as described below. The disc excision is typically conducted to a depth whereby the posterior margin of the disc prosthesis is located about 5 mm anterior to the posterior margin of the vertebral endplate. Alignment of the surgical instruments, such as the annulus incision guide (AIG), depth gauge (DG), disc space distractor (DSD), and the like, with respect to the external alignment frame (EAF) and the patient may be facilitated by the use of optically projected indicia, e.g., lines of light projected by laser alignment projectors, associated with, e.g., mounted on, the external alignment frame (EAF).

Following the adequate removal of the annulus and nucleus, a disc space distractor (DSD) (FIG. 5) is inserted into the prepared intervertebral space. The disc space distractor (DSD) is also set to a predetermined depth, essentially the same as that of the depth gauge. The disc space distractor (DSD) is set and aligned to its proper gross alignment position by reference to the guide crossbars of the external alignment frame (EAF). An adequate amount of distraction can be achieved by sequential distraction of the disc space using increasing sizes of distractor. Then the position and alignment of the disc space distractor (DSD) are confirmed by antero-posterior (AP) and lateral x-rays. The disc space distractor (DSD) has an alignment guidance system for x-rays, and further fine adjustment may be made according to the x-ray findings. Once the positioning and alignment of the disc space distractor (DSD) is satisfactory, vertebral guidance pins (VGPs) are inserted. An anterior midline notch on the anterior margin of the vertebral body is cut, if a prosthesis having endplates with generally vertical positioning and fixation fins, such as illustrated in FIG. 8a is to be implanted, and the distractor is removed leaving the vertebral guidance pins (VGPs) in place.

An endplate drill guide (EDG) (FIG. 6) is attached to the vertebral guidance pins (VGPs) and the vertebral endplate is reamed to the predetermined dimensions, i.e., size, shape and depth, which are exactly identical to those of the endplates of the disc prosthesis to be inserted. The specially designed vertebral endplate drill (VED) fits into the endplate drill guide (EDG).

Two posterior endplate cuts may be made by guidance of the vertebral guidance pins (VGPs) and the anterior midline notch, in order to accommodate a prosthesis endplate, such as illustrated in FIG. 8a, having posterior positioning and fixation fins.

The disc prosthesis is then clamped by the disc prosthesis holder (DPH) and inserted into the prepared disc space. Finally, the vertebral guidance pins (VGPs) are removed.

The surgical procedures following the implantation of the disc, e.g., closure and post-operative procedures, are generally conventional.

Description of the Instruments:

In the following description of the instruments of the invention used in the surgical procedure of the invention, the reference numerals are indicate the same elements throughout.

FIG. 1 illustrates the external alignment frame (EAF). An operating table 100 is provided with an external alignment frame 103. The external alignment frame 103 includes a generally U-shaped support 112 having a horizontal bar 108 supported by vertical legs 101, 102 fixed, one to each side, of the operating table 100 in a non-sterile field away from the operative field. An alignment reference bar 110, supported by U-shaped support 112 comprises an alignment “cross” 104, including a horizontal bar 105 and a vertical bar 106 meeting at right angles, and a telescoping right angle extension bar 107 that is secured generally at the center of the horizontal bar 108 of the U-shaped support 112. The portion of the extension bar 107 toward the operative field and the cross 104 are in the sterile field. The extension bar 107 can be telescoped to move the alignment cross 104 away from or closer to the operative field during the operation, as indicated by the double arrow 109. A second identical alignment reference bar 110 with extension bar 107 is kept not sterile and is used for positioning the patient on the operating table before the operation, thus setting up a midline reference. Optionally, laser line projectors may be attached to one or both of the crossbar 105 and extension bar 107, as shown at 113 and 114, respectively, to project visible lines onto the patient and/or instruments for accurate positioning of the instruments with respect to the relevant anatomical structures of the patient.

FIG. 2 illustrates the annulus incision guide (AIG) 200 of the invention.

The annulus incision guide (AIG) has three main parts, a handle 201, a shaft 204, and an incision guide plate 205. The handle 201 is equipped with horizontal bars 202, 203 set at right angles to each other and to the handle 201, forming a cross 206 for alignment with the “cross” 104 of the external alignment frame (EAF) of FIG. 1 as described above, in order to orient the handle 201 and attached shaft 204 vertically. An incision guide plate 205 is mounted on the distal end of shaft 204, preferably with an offset attachment fixture 207 as shown. The incision guide plate 205 has a general curvature matching that of the anterior edge of the intervertebral disc. The incision guide plate 205 is provided in various widths corresponding to different sizes of the intervertebral disc prosthesis. Once the crossbars 202, 203 and handle 201 of the annulus incision guide 200 (AIG) are aligned to the alignment cross 104 of the external alignment frame 112, the incision guide is properly aligned to the patient midline.

FIGS. 3a and 3b illustrate the depth gauge (DG) 300 of the invention.

The depth gauge (DG) 300 includes a number of component parts. A handle 304 is fixed to a proximal end of a shaft 302. A disc-shaped gauge block 301 (also sometimes herein, and particularly in FIG. 9, referred to as a “puck”), having a shape in the transverse body plane generally similar to that of the natural intervertebral disc (and the prosthesis) is attached at its anterior (or proximal) edge to the distal end of shaft 302. A gauge block 301 of an appropriate predetermined size is selected based on the measurement obtained by preoperative CT scan. The antero-posterior dimension of the disc-shaped block (or puck) 301 is indicated as L1 in FIGS. 3a and 3b. Representative examples of the disc-shaped gauge block 301 are shown in FIG. 9. The handle 304 is provided with cross bars 305, 306, which, by reference to the alignment cross 104 of the external reference frame 112, permit the depth gauge to be oriented vertically on the patient midline, by the same procedure as described above for the annulus incision guide 200. A housing 303, having a stop plate 310 at its distal end, is free to move along the shaft 302. In use, the depth gauge 300 is oriented generally vertically over the patient after at least a portion of the degenerated intervertebral disc has been excised. The gauge block (puck) 301 is inserted into the incised intervertebral cavity and the housing 303 is moved distally until the stop plate 304 contacts the anterior surface of the vertebra, as shown in FIG. 3b. The distance between the anterior edge of the gauge block 301 and the front edge of the spinal motion segment, indicated as L2 in FIGS. 3a and 3b, can then be determined from calibrated markings scribed on the shaft 302, generally indicated at 307. The incision can thus be controlled to provide the proper antero-posterior position of the prosthesis. In an alternative embodiment of the depth gauge 300, illustrated in FIGS. 3c and 3d, the shaft 302 can be threaded and provided with a threaded collar 308 that can be positioned at a predetermined position along the shaft 302. As shown in FIG. 3d, the threaded collar 308 can be adjusted on the shaft 302 to position the stop plate 310 of the housing 303 at a predetermined distance L2 from the anterior edge of the gauge block 301 when the proximal end of the housing 303 is in contact with the collar 308. In this embodiment, based on the length of the housing 303 (indicated as L3 in FIGS. 3c and 3d) and the antero-posterior dimension of the gauge block 301, the collar 308 can be set to position the disc-shaped gauge block 301 at the proper location within the intervertebral space when the stop plate 310 is in contact with the anterior surfaces of the vertebrae of the spinal motion segment. This embodiment of the depth gauge 300 can be inserted into the incision cavity periodically during the surgical procedure to determine whether the cavity has been made deep enough for the selected prosthesis. FIG. 9 includes Table 1 illustrating appropriate dimensions for insertion and positioning of the disc-shaped block 1.

FIGS. 4a and 4b illustrate a positioning/alignment block 401 for mounting the endplate drill guide 600 on a vertebra of the spinal motion segment and the installation of the positioning/alignment block onto a vertebra of the spinal motion segment.

A positioning/alignment block (PAB) 401 (seen in a lateral partial section and partial phantom view in FIG. 4a) is secured to an alignment plate 402, by means of a fixation flange 406. The alignment plate 402 is inserted into the prepared intervertebral space 403 and positioned in contact with the vertebra to position the positioning/alignment block (PAB) in a proper position for attachment to the vertebral body 411. One or more fixation pins (or vertebral guidance pins (VGPs)) 410 are inserted into the vertebral body 411 in positions to mate with holes 408 in the PAB. The PAB has a fixation pin-holding fixture 404 whereby an inserted fixation pin (or vertebral guidance pin (VGP)) 410 that is secured to the vertebral body 411 will also be secured to the positioning/alignment block (PAB). This assembly allows for alignment of the vertebral endplate face 405 to the positioning/alignment block (PAB) and, by means of one or more of the fixation pins 410, secures the positioning/alignment block (PBA) in a fixed reference position. This fixed reference position is utilized to provide alignment of subsequently used instruments with respect to the vertebral endplate face 405 in all planes and axes. The template 402 is removably attached to the positioning/alignment block (PAB), and thus may be removed to expose the vertebral endplate face 405 for subsequent operations. FIG. 4b shows an elevational view of the positioning/alignment block 401 taken in the direction 4b-4b in FIG. 4a. The fixation pin holding fixture 404 comprises cam knobs 407 acting on flexible portions 412 to reduce the diameters of fixation pin holes 408, thus capturing the fixation pins 410. Knob 409 is used to secure and release the template 402, as well as to secure subsequently used instruments.

FIGS. 5a-5h illustrate a disc space distractor usable in the disc replacement procedure of the invention.

The disc space distractor (DSD) 500 is provided with a handle 501. The DSD 500 has a generally cylindrical shaft 508 and a flat end portion 502. The distraction device has two generally parallel distractor plates 503, 504, preferably made of an appropriate metal, that are connected to a tube 405 (preferably metal) in such a way that the two plates will be separated when the distraction mechanism is operated. In the illustrated embodiment, the plates 503, 504 are cut away, as indicated at 516, to leave relatively narrow strips 506, 507 at the edges thereof to facilitate deformation during distraction as shown in cross-section in FIG. 5f. Although not specifically illustrated in the drawings, the broad dimension of the flat end portion tip 502 preferably varies along the lengthwise direction of the end portion 502, being somewhat narrower toward the distal end of the end portion 502. Thus, in a preferred embodiment, the ratio of thickness to width of the end portion 502 varies along its length. In use, the end portion 502 is initially inserted through the hollow tube 505 and between the distractor plates 503, 504, with the broad dimension of the tip generally parallel to the planes of the parallel plates as shown in FIG. 5b (end elevation view in direction 5b as indicated in FIG. 5d). The initial distance between the plates 502, 503 is indicated as H₁ in FIG. 5b. When the cylindrical shaft 508 with flat end portion 502 is turned 90 degrees, the distractor plates 503, 504 will be distracted from the original height H₁ to the final height H₁+H₂ (see FIG. 5c, end elevation view in direction 5b as indicated in FIG. 5d). A wide range of distraction can be accomplished by using different ratios of thickness/width of the flat end portion 502 of the shaft 508. The distractor plates 503, 504 may also provide variable amounts of distraction depending on the rotational angular position of the flat end portion 502 of the shaft 508 within the disc space. Furthermore, as shown in FIGS. 5e and 5f (wherein the letters P and A indicate posterior and anterior locations with respect to the vertebra, when the disc space distractor DSD is in use in surgery) when the tip 509 of the flat end portion 502 of the shaft 508 is positioned somewhat anteriorly to the ends of the plates 503, 504, there will be more anterior distraction than posterior since, as illustrated in FIG. 5f, the more posterior force F_{P} will operate with greater effect than the more anterior force F_{A}.

FIG. 5g shows an optional addition to the disc space distractor (DSD) 500. A spacer block, e.g., a parallel spacer block 510, having an outer surface generally parallel to the outer surface of the distractor plates, e.g., distractor plate 503 as illustrated in FIG. 5g, may be removably fitted onto one or both of the distractor plates, 503, 504, for example with a dovetail fitting 511. By sliding a spacer block 510 having parallel faces onto the disc space distractor the net initial height, as well as the final height, is increased accordingly. Angled spacer blocks 512 of various inclinations can also be used to pre-configure the distraction angle as shown in FIG. 5h.

FIGS. 6a-6f illustrate an endplate drill guide (EDG) 600 used in preparing the vertebral endplates to receive an intervertebral disc prosthesis.

The endplate drill guide (EDG) 600 is designed to provide positive control of the movement of a cutter that abrades the vertebral endplate to provide a surface shape or profile that conforms to the shape or profile of the disc prosthesis. Accordingly, the vertebral endplate is given a generally concave surface that matches the generally convex outer surface of the upper or lower mating surface of a disc prosthesis. Such a concave surface of a vertebral endplate is characterized by radii of curvature in the anterior-posterior (sagittal) plane and in the left-right (coronal) plane. A coordinate system for describing these radii and the method of producing them using the endplate drill guide (EDG) of the invention is illustrated in FIG. 6a. Thus, FIG. 6a illustrates a coordinate system wherein the transverse axis is defined as the x-axis, the vertical or longitudinal axis of the spine is defined as the y-axis, and the anterior-posterior axis is defined as the z-axis. The illustration thus shows a coronal (transverse) plane as the xy-plane and a sagittal (anterior-posterior) plane as the yz-plane. The radii of curvature in the mutually perpendicular xy- and yz-planes are shown as R_{yx} and R_{yz}, respectively.

A generally perspective view of the endplate drill guide (EDG) 600 with the vertebral endplate cutter (VEC) 628 mounted thereon is shown in FIG. 6f. FIG. 6b shows a schematic lateral elevation of the endplate drill guide (EDG) 600. FIG. 6c shows a somewhat enlarged elevational end view taken in the direction and location indicated by the line 6c-6c in FIG. 6b. FIG. 6d is a somewhat enlarged detail lateral elevational view of the elements indicated by the circle 6d in FIG. 6b. FIG. 6e shows a plan view of the xz-plane limiter template element described in more detail below. The endplate drill guide (EDG) 600 may be considered to have five functional subunits. The first subunit is a positioning and mounting subunit 620 that is fixed to the positioning/alignment block (PAB) 500 and provides a base and position reference for the endplate drill guide (EDG) 600. The second subunit is an xz-plane limiter template 621, which is fixed to the positioning and mounting subunit 620 and also provides structure 632 for mounting and positioning the third subunit with respect to the positioning and mounting subunit 620. The lower portion of structure 632 forms link 601 of subunit 622. The third subunit is an xy-plane guide linkage 622, which includes a classic four bar linkage with links 602 and 604 supported on link 601, which are parallel and of equal length, operable in the xy-plane. Link 603 of the third subunit (xy-plane guide linkage 622) provides a base for the fourth subunit, which is a yz-plane guide linkage 623. The fourth subunit (yz-plane guide linkage 623) also includes a four bar linkage similar to that of the third subunit (xy-plane guide linkage 622) and is operable in the yz-plane. The linkage 623 is formed of links 604 and 605, which are parallel and of equal length, mounted on link 603 and operable in the yz-plane. The fifth subunit is a cutting tool 624 fixed to link 605 of the yz-plane guide linkage, extending in the negative z-direction and positioned to mill a bi-curvature dome cavity in a vertebral endplate with reference to the positioning/alignment block (PAB) 500. A limit pin 625, mounted on the cutting tool 624, is positioned within the template 626 to provide travel limits for the cutting tool 624 in the xz-plane and the xy-plane. When the tool is moved in the xz-plane and xy-plane, the third and fourth subunits, i.e., the xy-plane guide linkage 22 and the yz-plane guide linkage 623, by virtue of their construction, force the cutting tool tip 627 to move on a surface defined by the arcs R_{yz} and R_{yx}. The actual radii formed by the cutting edges of the tool are defined by the effective length of links 602, 604 and 606, 607, respectively, and the actual diametric size of the cutter or burr 628 at the cutting tool tip 627. The template size is determined by an offset equal to half the diameter of the cutter or burr 628 plus the diameter of the limit pin 625.

FIGS. 7a-7b illustrate the vertebral endplate cutter 628 of the invention.

The vertebral endplate cutter 628 (VEC) is the cutter or burr mounted on the cutting tool 624 of the endplate drill guide (EDG) 600. The cutting surface of the vertebral endplate cutter (VEC) 628 has a generally domed shape with a diameter D and a dome radius R_{T}, which is less than the radius of the concave surface to be cut in the vertebral endplate. The cutting tool 624 provides a right-angle drive configuration whereby rotation of a drive shaft having a rotation axis parallel to the z-axis of the endplate drill guide (EDG) 600 rotates the cutter or burr 628 in a rotational axis parallel to the y-axis. When the endplate drill guide (EDG) 600 is operated, the vertebral endplate cutter (VEC) 628 undergoes translational motion in the xy-and yz-planes, but is forced, by the xy-plane guide linkage 622 and the yz-plane guide linkage 623, to follow an arc, e.g., in the xy-plane, of radius R₂, where R₂ is the length of the corresponding link of the endplate drill guide (EDG) 600. However, the vertebral endplate cutter (VEC) 628 cuts an arc of radius R₁ as illustrated in FIG. 7a, since the rotational axis of the vertebral endplate cutter (VEC) 628 remains parallel to the y-axis. This allows for the reduction of the length of the links of the corresponding guide linkage mechanism, since the cutting diameter of the VEC produces a concave surface having a diameter of R₁, which is greater than R₂. The vertical endplate cutter (VEC) 628 dome radius R_{T} cannot be greater than the smallest of the bi-curvature radii of the concave surfaces to be cut into the vertebral endplate. The resultant bi-curvature radii are related to the diameter of the cutter or burr (VEC) 628. For example, increasing the diameter of the vertebral endplate cutter (VEC) 628 will increase the radii of the curvatures formed by the vertebral endplate cutter (VEC) 628.

FIGS. 8a-8c illustrate the disc prosthesis holder (DPH) 800 of the invention.

The disc prosthesis holder (DPH) 800 is designed to firmly, but releasably, grip an intervertebral prosthesis for accurate and convenient implantation. FIG. 8a shows a generally perspective view of the holder gripping an intervertebral prosthesis 850. The illustrated embodiment of the disc prosthesis holder comprises a shank 840, having a gripping end 841 and a locking collar 842, which is slidably mounted on the shank 840, and can be moved toward or away from the gripping end 841, as indicated by the double arrow 843. The gripping end 841 of the disc prosthesis holder (DPH) 800 has a profile that closely matches the shape of the anterior edge of the implant and has protruding tips 844 that engage corresponding cavities on the implant. The gripping end 841 is split crosswise to provide four somewhat flexible times 845. The structure of the gripping end may be seen in the cross-sectional views of FIG. 8b, taken along the line 8b-8b in FIG. 8a, and FIG. 8c, taken along line 8c-8c in FIG. 8a. The gripping end 841 of the disc prosthesis holder (DPH) 800 has an external tapered surface such that when the locking collar 842 is moved toward the gripping end 841 the times 845 are forced together and thereby grip the implant. Retraction of the locking collar 842 allows the times 845 to spring back to their open position, thus releasing the implant.

The invention having been described above in terms of certain embodiments, it will be apparent to those skilled in the art that many changes and alterations can be made without departing from the spirit or essential characteristics of the invention. The present disclosure is therefore to be considered as illustrative, and not restrictive, of the invention.

We claim:

1. A vertebral endplate drill guide for preparing a vertebral endplate of a vertebra for contact with an intervertebral prosthesis, comprising:
a mounting block adapted to be secured to an anterior surface of a vertebra of a spinal motion segment;
a cutting device including
a cutter adapted to prepare said vertebral endplate for contact with said intervertebral prosthesis,
a driving mechanism to drive said cutter, and
a limit member mounted on said cutting device;
a transverse shape control element extending generally anteriorly and horizontally from said mounting block and including a horizontal template aperture disposed and shaped to cooperate with said limit member to limit motion of said cutter in a horizontal plane; and
a link mechanism coupled to said transverse shape control element and said cutting device and supporting said cutting device for rocking movement in each of a coronal plane and a sagittal plane.

2. The vertebral endplate drill guide of claim 1 wherein said link mechanism includes a four-bar linkage oriented to limit motion of said cutter in a coronal plane.

3. The vertebral endplate drill guide of claim 1 wherein said link mechanism includes a four-bar linkage oriented to limit motion of said cutter in a sagittal plane.

4. The vertebral endplate drill guide of claim 1 wherein said link mechanism includes a four-bar linkage oriented to limit motion of said cutter in a coronal plane and a four-bar linkage oriented to limit motion of said cutter in a sagittal plane.

5. A depth gauge for evaluating the size of an intervertebral incision, comprising:
a gauge block, shaped and sized to represent a cross-section of an intervertebral prosthesis;
a support rod, having an axis, adapted to support said gauge block at a distal end of said support rod;
a housing slidably mounted on said support rod and having a stop plate at its distal end oriented generally perpendicular to said axis of said support rod;
said support rod having indicia for indicating the position of said stop plate with respect to said gauge block.

6. The depth gauge of claim 5, additionally comprising a handle fastened to said support rod at its proximal end.

7. The depth gauge of claim 6, wherein said handle is provided with at least one crossbar oriented generally at right angles to said support rod and adapted to be oriented with respect to an external reference standard.

8. The depth gauge of claim 6, wherein said handle is provided with a pair of crossbars oriented generally at right angles to said support rod and to each other adapted to be oriented with respect to an external reference standard.

9. A depth gauge for evaluating the size of an intervertebral incision, comprising:
a gauge block, shaped and sized to represent a cross-section of an intervertebral prosthesis;
a threaded support rod, having an axis, adapted to support said gauge block at a distal end of said support rod;
a housing slidably mounted on said support rod and having a stop plate at its distal end oriented generally perpendicular to said axis of said support rod;
a threaded collar positioned on said support rod proximal of said housing and adapted to contact a proximal end of said housing to position said stop plate a predetermined distance from said gauge block.

10. The depth gauge of claim 9, additionally comprising a handle fastened to said support rod at its proximal end.

11. The depth gauge of claim 10, wherein said handle is provided with at least one crossbar oriented generally at right angles to said support rod and adapted to be oriented with respect to an external reference standard.

12. The depth gauge of claim 10, wherein said handle is provided with a pair of crossbars oriented generally at right angles to said support rod and to each other adapted to be oriented with respect to an external reference standard.

13. An external alignment frame for aligning surgical instruments in a surgical procedure for excising an interveretbral disc and implanting an intervertebral disc prosthesis, comprising:
an extension rod adapted to be supported horizontally and longitudinally above a surgical operating table, said extension rod having at a first end thereof at least one crossbar oriented generally at right angles to said support rod; and
a frame for supporting said extension rod above said surgical operating table.

14. The external alignment frame of claim 13, wherein said extension bar has at said first end thereof a pair of crossbars oriented generally at right angles to said support rod and to each other.

15. The external alignment frame of claim 13, additionally comprising a laser indicia projector mounted on said at least one crossbar to project indicia onto a surgical field.

16. The external alignment frame of claim 14, additionally comprising laser indicia projectors mounted on each of said crossbars to project indicia onto a surgical field.

17. A disc space distractor for distracting adjacent vertebrae of a spinal motion segment, comprising:
an elongated distraction member comprising a tubular portion and a pair of elongated plates extending axially from a first end of said tubular portion, mounted radially opposite to one another and oriented generally parallel to one another;
an elongated actuating shaft housed within said distraction member, said shaft having a cylindrical portion housed within said tubular portion of said distraction member and a flat portion extending from an end of said cylindrical portion between said pair of elongated plates,
said flat portion of said actuating shaft having a thickness and a lateral dimension such that when said flat portion is oriented parallel to said elongated plates, said plates are spaced closely enough to be inserted between adjacent vertebrae of a spinal motion segment and when said flat portion is disposed generally perpendicularly to said elongated plates said elongated plates are separated to distract said adjacent vertebrae.

18. The disc space distractor of claim 17, additionally comprising at least one spacer block removably fited onto at least one of said elongated plates.
